(19) United States
(12) Patent Application Publication
(10) Pub. No.: US 2025/0099468 A1
(43) Pub. Date: Mar. 27, 2025

(54) TREATING LIVER DISORDERS WITH AN SSAO INHIBITOR

(71) Applicant: Terns Pharmaceuticals, Inc., Foster City, CA (US)

(72) Inventors: Christopher T. JONES, Foster City, CA (US); Martijn FENAUX, San Mateo, CA (US); Yujin WANG, San Jose, CA (US); Feng JIN, Foster City, CA (US); D. Barry CRITTENDEN, Foster City, CA (US); Erin K. QUIRK, Foster City, CA (US)

(21) Appl. No.: 18/976,460

(22) Filed: Dec. 11, 2024

Related U.S. Application Data
(63) Continuation of application No. 17/985,740, filed on Nov. 11, 2022.
(60) Provisional application No. 63/349,978, filed on Jun. 7, 2022.
(60) Provisional application No. 63/263,934, filed on Nov. 11, 2021.

(51) Int. Cl. A61K 31/506; A61K 31/355; A61K 31/454; A61K 31/55; A61K 31/575; A61K 31/732; A61K 38/05; A61P 1/16

(52) U.S. Cl. CPC A61K 31/506; A61K 31/355; A61K 31/454; A61K 31/55; A61K 31/575; A61K 31/732; A61K 38/05; A61P 1/16

(57) ABSTRACT

Provided herein are methods for treating liver disorders, including non-alcoholic steatohepatitis (NASH), and symptoms and manifestations thereof, in a patient comprising administration of the SSAO inhibitor:

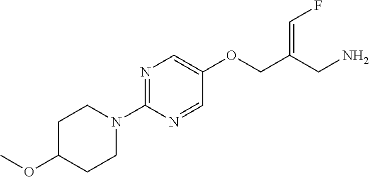 [SMILES: COC1CCN(c2ncc(OC/C(=C/F)CN)cn2)CC1]

or a pharmaceutically acceptable salt thereof.

### CROSS REFERENCE TO RELATED APPLICATIONS

This application is a continuation of U.S. application Ser. No. 17/985,740 filed Nov. 11, 2022, which claims the benefit of and priority to U.S. Provisional Application Nos. 63/263,934 filed on Nov. 11, 2021, and 63/349,978 filed on Jun. 7, 2022. The contents of the aforementioned patent applications are incorporated herein by reference in their entirety for all purposes.

### FIELD OF THE INVENTION

This invention relates to methods and compositions for treating liver disease including non-alcoholic steatohepatitis (NASH) in a patient.

### BACKGROUND

Fatty liver disease (FLD) encompasses a spectrum of disease states characterized by excessive accumulation of fat in the liver often accompanied with inflammation. FLD can lead to non-alcoholic fatty liver disease (NAFLD). If untreated, NAFLD can progress to a persistent inflammatory response or non-alcoholic steatohepatitis (NASH), progressive liver fibrosis, and eventually to cirrhosis. In Europe and the US, NAFLD is the second most common reason for liver transplantation. Accordingly, the need for treatment is urgent, but due to the lack of obvious symptoms to the patient, patients may lack the motivation to maintain treatment regimens, particularly burdensome treatment regimens, such as injected medicines, medications that are administered many times a day, or any that produce dangerous or irritating side effects. There is currently no approved treatment of NASH.

### BRIEF SUMMARY

Provided herein are methods and compositions for treating liver disorders such as NASH in a patient in need thereof. The methods comprise administering to the patient a Semicarbazide-Sensitive Amine Oxidase (SSAO)/Vascular Adhesion Protein-1 (VAP-1) inhibitor (referred to herein as “Compound 1”) having the following structure:

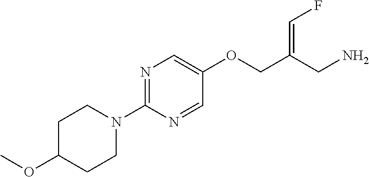 [SMILES: COC1CCN(c2ncc(OC/C(=C/F)CN)cn2)CC1]

or a pharmaceutically acceptable salt thereof. Compound 1, which has the chemical name (E)-3-fluoro-2-(((2-(4-methoxypiperidin-1-yl)pyrimidin-5-yl)oxy)methyl)prop-2-en-1-amine, was described in US Published Application No. 2018/0297987, which is incorporated herein by reference in its entirety. In one form, Compound 1 is provided as a free base. In other forms, Compound 1 is provided as an acid addition salt, such as a mono or di HCl addition salt(s) or a sulfonate salt, preferably a 4-methylbenzenesulfonate (a tosylate salt).

Inventors have discovered that Compound 1 or a pharmaceutically acceptable thereof, can be administered to patients suffering from liver disorders at surprisingly low doses while still maintaining the desired level of efficacy. This discovery is based, in part, on the observation that Compound 1 exhibits saturable-target mediated clearance owing to its very strong affinity for plasma SSAO. Compound 1, or a pharmaceutically salt thereof, can be administered at doses that achieve full saturation of plasma SSAO or less than full saturation of plasma SSAO. Plasma SSAO is the active, soluble form of plasma SSAO that is released into the plasma by membrane-bound plasma SSAO. As described herein, administration of Compound 1, or a pharmaceutically salt thereof, at doses less than saturation of plasma SSAO are still efficacious. In some embodiments, Compound 1, or a pharmaceutically acceptable salt thereof, can be administered at a dose that achieves at least about 25% saturation of the plasma SSAO target. In other embodiments, Compound 1, or a pharmaceutically acceptable salt thereof, can be administered at a dose that achieves at least about 50% saturation of the plasma SSAO target. In other embodiments, Compound 1, or a pharmaceutically acceptable salt thereof, can be administered at a dose that achieves at least about 75% saturation of the plasma SSAO target. In other embodiments, Compound 1, or a pharmaceutically acceptable salt thereof, can be administered at a dose that achieves at least about 95% saturation of the plasma SSAO target. In other embodiments, Compound 1, or a pharmaceutically acceptable salt thereof, can be administered at a dose that achieves about 100% saturation of the plasma SSAO target.

In some embodiments of the disclosure, Compound 1, or a pharmaceutically acceptable salt thereof, can be administered to patients orally once daily at doses as low as 1 mg or less and still sufficiently reduce amine oxidase activity and reduce lymphocyte adhesion and transmigration. For example, Compound 1, or a pharmaceutically acceptable salt thereof, can be administered orally once daily to a patient with a liver disorder (e.g. NASH) at a dose of from about 0.5 mg to about 25 mg. In some embodiments, Compound 1, or a pharmaceutically acceptable salt thereof, can be administered orally once daily to a patient with a liver disorder (e.g. NASH) at a dose of from about 1 mg to about 15 mg. In some embodiments, Compound 1, or a pharmaceutically acceptable salt thereof, can be administered orally once daily to a patient with a liver disorder (e.g. NASH) at a dose of from about 2 mg to about 10 mg. In some embodiments, Compound 1, or a pharmaceutically acceptable salt thereof, can be administered orally once daily to a patient with a liver disorder (e.g. NASH) at a dose of about 1 mg. In some embodiments, Compound 1, or a pharmaceutically acceptable salt thereof, can be administered orally once daily to a patient with a liver disorder (e.g. NASH) at a dose of about 4 mg. In some embodiments, Compound 1, or a pharmaceutically acceptable salt thereof, can be administered orally once daily to a patient with a liver disorder (e.g. NASH) at a dose of about 5 mg. In some embodiments, Compound 1, or a pharmaceutically acceptable salt thereof, can be administered orally once daily to a patient with a liver disorder (e.g. NASH) at a dose of about 10 mg. In some embodiments, Compound 1, or a pharmaceutically acceptable salt thereof, can be administered orally once daily to a patient with a liver disorder (e.g. NASH) at a dose of about 20 mg.

Moreover, inventors have surprisingly discovered that Compound 1 has a prolonged pharmacodynamics effect which supports dosing to patients with liver disorders (e.g., NASH) at even less than once daily. For instance, Compound 1, or a pharmaceutically acceptable salt thereof, can be dosed once every other day (e.g., three or four times weekly). In some embodiments, Compound 1, or a pharmaceutically acceptable salt thereof, can be dosed once weekly. In embodiments where Compound 1, or a pharmaceutically acceptable salt thereof, is administered less than once daily, administration may be orally or parenterally (e.g., intramuscularly, subcutaneously, or intravenously).

In some embodiments of the present disclosure, Compound 1, or a pharmaceutically acceptable salt thereof, is administered to a patient suffering from a liver disorder (e.g., NASH) at a dose that that results in methylamine concentrations of at least 25 ng/mL in the plasma when Compound 1 reaches its steady state concentration in the plasma. In other embodiments, Compound 1, or a pharmaceutically acceptable salt thereof, is administered to a patient suffering from a liver disorder (e.g., NASH) at a dose that that results in methylamine concentrations of at least about 50 ng/ml in the plasma when Compound 1 reaches its steady state concentration in the plasma. In other embodiments, Compound 1, or a pharmaceutically acceptable salt thereof, is administered to a patient suffering from a liver disorder (e.g., NASH) at a dose that that results in methylamine concentrations of at least 100 ng/mL in the plasma when Compound 1 reaches its steady state concentration in the plasma. In other embodiments, Compound 1, or a pharmaceutically acceptable salt thereof, is administered to a patient suffering from a liver disorder (e.g., NASH) at a dose that that results in methylamine concentrations of at least about 100 ng/ml in the plasma when Compound 1 reaches its steady state concentration in the plasma. In other embodiments, Compound 1, or a pharmaceutically acceptable salt thereof, is administered to a patient suffering from a liver disorder (e.g., NASH) at a dose that that results in methylamine concentrations of at least about 200 ng/mL in the plasma when Compound 1 reaches its steady state concentration in the plasma. In other embodiments, Compound 1, or a pharmaceutically acceptable salt thereof, is administered to a patient suffering from a liver disorder (e.g., NASH) at a dose that that results in methylamine concentrations of at least about 300 ng/mL in the plasma when Compound 1 reaches its steady state concentration in the plasma. In other embodiments, Compound 1, or a pharmaceutically acceptable salt thereof, is administered to a patient suffering from a liver disorder (e.g., NASH) at a dose that that results in methylamine concentrations of at least about 300 ng/mL in the plasma when Compound 1 reaches its steady state concentration in the plasma. In other embodiments, Compound 1, or a pharmaceutically acceptable salt thereof, is administered to a patient suffering from a liver disorder (e.g., NASH) at a dose that that results in methylamine concentrations of from about 25 ng/ml to about 400 ng/ml in the plasma when Compound 1 reaches its steady state concentration in the plasma. In other embodiments, Compound 1, or a pharmaceutically acceptable salt thereof, is administered to a patient suffering from a liver disorder (e.g., NASH) at a dose that that results in methylamine concentrations of from about 50 ng/ml to about 350 ng/mL in the plasma when Compound 1 reaches its steady state concentration in the plasma. In other embodiments, Compound 1, or a pharmaceutically acceptable salt thereof, is administered to a patient suffering from a liver disorder (e.g., NASH) at a dose that that results in methylamine concentrations of from about 100 ng/ml to about 300 ng/mL in the plasma when Compound 1 reaches its steady state concentration in the plasma.

In one aspect, the disclosure provides methods of reducing hepatic inflammation in a patient in need thereof, comprising administering to the patient Compound 1, or a pharmaceutically acceptable salt thereof, wherein Compound 1, or a pharmaceutically acceptable salt thereof, is administered at a dose as described herein.

In another aspect, the disclosure provides methods of reducing hepatic fibrosis in a patient in need thereof, comprising administering to the patient Compound 1, or a pharmaceutically acceptable salt thereof, wherein Compound 1, or a pharmaceutically acceptable salt thereof, is administered at a dose as described herein.

In another aspect, the disclosure provides methods of treating a disease or condition characterized by fibrosis of the liver, comprising administering to the patient Compound 1, or a pharmaceutically acceptable salt thereof, wherein Compound 1, or a pharmaceutically acceptable salt thereof, is administered at a dose as described herein.

### BRIEF DESCRIPTION OF THE FIGURES

FIG. 1A shows the plasma SSAO-specific amine oxidase activity compared to baseline of healthy volunteers administered a single dose of placebo or 1, 3, 6, or 10 mg of Compound 1 at 4 hours and 168 hours post dose. FIG. 1B shows a time course of plasma total amine oxidase activity compared to baseline of healthy volunteers administered a single dose of placebo or 1, 3, 6, or 10 mg of Compound 1. FIG. 1C shows a time course of the level of Compound 1 after a single dose of placebo or 1, 3, 6, or 10 mg in healthy volunteers. FIG. 1D shows a time course of plasma methylamine concentration after a single dose of placebo or 1, 3, 6, or 10 mg of Compound 1 in healthy volunteers.

FIG. 2A shows a time course of the concentration of Compound 1 after a single dose of 1, 4, or 10 mg of Compound 1 in healthy volunteers. FIG. 2B shows a time course of the concentration of Compound 1 on the last day of 7 daily doses of 1 mg or 4 mg of Compound 1, and the last day of 14 daily doses of 10 mg of Compound 1.

FIG. 3A shows SSAO-specific amine oxidase activity 24 hours after a first daily dose of 1, 4, or 10 mg Compound 1. FIG. 3B shows a time course of plasma methylamine concentration after a first daily dose of 1, 4, or 10 mg Compound 1. FIG. 3C shows a time course of plasma methylamine concentration after the last day of 7 daily doses of 1 mg or 4 mg of Compound 1, and the last day of 14 daily doses of 10 mg of Compound 1. FIG. 3D shows a time course of total amine oxidase activity after a single dose of 1, 4, or 10 mg of Compound 1 in healthy volunteers. FIG. 3E shows a 14 day time course of total amine oxidase activity after the 6th of 7 daily doses of 1 mg or 4 mg of Compound 1, or the 13th of 14 daily doses of 10 mg of Compound 1, in healthy volunteers.

FIG. 4 shows the overall study design for Compound 1.

FIG. 5 shows patient demographics and baseline characteristics.

FIG. 6 shows the patient disposition according to the study design.

FIG. 7 shows overall summary of adverse events.

FIG. 8A shows TIMP-1 change from baseline to week 6 and week 12.

FIG. 8B shows results regarding NASH and inflammation biomarkers mean (SE) change from baseline to week 12.

FIG. 9 shows cT1 changes in week 12.

FIG. 10 shows VAP-1/SSAO activity at week 12 relative to baseline, %.

FIG. 11 shows ELF components (TIMP-1, P3NP, HA) change from baseline to week 12.

FIG. 12 shows change in ICAM-1 and VCAM-1 from baseline to week 12.

### DETAILED DESCRIPTION

### Definitions

As used herein, the following definitions shall apply unless otherwise indicated. Further, if any term or symbol used herein is not defined as set forth below, it shall have its ordinary meaning in the art.

“Comprising” is intended to mean that the compositions and methods include the recited elements, but not exclude others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this invention.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Patient” refers to mammals and includes humans and non-human mammals. Examples of patients include, but are not limited to mice, rats, hamsters, guinea pigs, pigs, rabbits, cats, dogs, goats, sheep, cows, and humans. In some embodiments, patient refers to a human.

“Pharmaceutically acceptable” refers to safe and non-toxic, preferably for in vivo, more preferably, for human administration.

“Pharmaceutically acceptable salt” refers to a salt that is pharmaceutically acceptable. A compound described herein may be administered as a pharmaceutically acceptable salt.

“Salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionality, such salts include, without limitation, salts of organic acids, such as carboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the likes.

“Therapeutically effective amount” or dose of a compound or a composition refers to that amount of the compound or the composition that results in reduction or inhibition of symptoms or a prolongation of survival in a patient. The results may require multiple doses of the compound or the composition.

“Treatment” or “treating” refers to an approach for obtaining beneficial or desired results including clinical results. For purposes of this invention, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the invention contemplate any one or more of these aspects of treatment.

As used herein, “delaying” development of a disease means to defer, hinder, slow, retard, stabilize and/or postpone development of the disease and/or slowing the progression or altering the underlying disease process and/or course once it has developed. This delay can be of varying lengths of time, depending on the history of the disease and/or individual being treated. As is evident to one skilled in the art, a sufficient or significant delay can, in effect, encompass prevention, in that the individual does not develop clinical symptoms associated with the disease. A method that “delays” development of a disease is a method that reduces probability of disease development in a given time frame and/or reduces extent of the disease in a given time frame, when compared to not using the method, including stabilizing one or more symptoms resulting from the disease.

An individual who is “at risk” of developing a disease may or may not have detectable disease, and may or may not have displayed detectable disease prior to the treatment methods described herein. “At risk” denotes that an individual has one or more so-called risk factors, which are measurable parameters that correlate with development of a disease. An individual having one or more of these risk factors has a higher probability of developing the disease than an individual without these risk factor(s). These risk factors include, but are not limited to, age, sex, race, diet, history of previous disease, presence of precursor disease and genetic (i.e., hereditary) considerations. Compounds may, in some embodiments, be administered to a subject (including a human) who is at risk or has a family history of the disease or condition.

The dosage amount of a compound as described herein is determined based on the free base of a compound.

### Pharmaceutically Acceptable Compositions and Formulations

Pharmaceutically acceptable compositions or simply “pharmaceutical compositions” of any of the compounds detailed herein are embraced by this invention. Thus, the invention includes pharmaceutical compositions comprising Compound 1, or a pharmaceutically acceptable salt thereof, and a pharmaceutically acceptable carrier or excipient. In some embodiments, the pharmaceutically acceptable salt is an acid addition salt, such as a salt formed with an inorganic or organic acid. Pharmaceutical compositions according to the invention may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound intends a composition that contains no more than 35% impurity, wherein the impurity denotes a compound other than the compound or a salt thereof. In one variation, a composition of substantially pure compound or a salt thereof is provided wherein the composition contains no more than 25% impurity. In another variation, a composition of substantially pure compound or a salt thereof is provided wherein the composition contains or no more than 20% impurity. In still another variation, a composition of substantially pure compound or a salt thereof is provided wherein the composition contains or no more than 10% impurity. In a further variation, a composition of substantially pure compound or a salt thereof is provided wherein the composition contains or no more than 5% impurity. In another variation, a composition of substantially pure compound or a salt thereof is provided wherein the composition contains or no more than 3% impurity. In still another variation, a composition of substantially pure compound or a salt thereof is provided wherein the composition contains or no more than 1% impurity. In a further variation, a composition of substantially pure compound or a salt thereof is provided wherein the composition contains or no more than 0.5% impurity. In yet other variations, a composition of substantially pure compound means that the composition contains no more than 15% or preferably no more than 10% or more preferably no more than 5% or even more preferably no more than 3% and most preferably no more than 1% impurity, which impurity may be the compound in a different stereochemical form.

In one variation, the compounds herein are synthetic compounds prepared for administration to an individual such as a human. In another variation, compositions are provided containing a compound in substantially pure form. In another variation, the invention embraces pharmaceutical compositions comprising a compound detailed herein and a pharmaceutically acceptable carrier or excipient. In another variation, methods of administering a compound are provided. The purified forms, pharmaceutical compositions and methods of administering the compounds are suitable for any compound or form thereof detailed herein.

The compounds may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

Compounds described herein can be used in the preparation of a formulation, such as a pharmaceutical formulation, by combining the compounds as active ingredients with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21^{st} ed. (2005), which is incorporated herein by reference.

Compounds as described herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid polyols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described herein.

The present disclosure further encompasses kits (e.g., pharmaceutical packages). The kit provided may comprise the pharmaceutical compositions or the compounds described herein and containers (e.g., drug bottles, ampoules, bottles, syringes and/or subpackages or other suitable containers). In some embodiments, the kit includes a container comprising Compound 1, or a pharmaceutically acceptable salt thereof.

In some embodiments, provided herein is a dosage form comprising a therapeutically effective amount of Compound 1, or a pharmaceutically acceptable salt thereof.

### Methods of Use and Uses

Compounds and compositions described herein may in some aspects be used in treatment or prevention of liver disorders. In some embodiments, the method of treating or preventing a liver disorder in a patient Compound 1, a pharmaceutically acceptable salt thereof.

Liver disorders include, without limitation, liver inflammation, fibrosis, and steatohepatitis. In some embodiments, the liver disorder is selected from liver inflammation, liver fibrosis, alcohol induced fibrosis, primary sclerosing cholangitis (PSC), primary biliary cirrhosis (PBC), non-alcoholic fatty liver disease (NAFLD), and non-alcoholic steatohepatitis (NASH). In certain embodiments, the liver disorder is selected from: liver fibrosis, alcohol induced fibrosis, NAFLD, and NASH. In one embodiment, the liver disorder is NASH. In another embodiment, the liver disorder is liver inflammation. In another embodiment, the liver disorder is liver fibrosis. In another embodiment, the liver disorder is alcohol induced fibrosis. In another embodiment, the liver disorder is NAFLD. In one embodiment, the treatment methods provided herein impedes or slows the progression of NAFLD to NASH. In one embodiment, the treatment methods provided herein impedes or slows the progression of NASH. NASH can progress, e.g., to one or more of liver cirrhosis, hepatic cancer, etc. In some embodiments, the liver disorder is NASH. In some embodiments, the patient has had a liver biopsy. In some embodiments, the method further comprising obtaining the results of a liver biopsy.

In some embodiments, the method of treating a liver disorder in a patient in need thereof, wherein the liver disorder is selected from the group consisting of liver inflammation, liver fibrosis, alcohol induced fibrosis, primary sclerosing cholangitis (PSC), primary biliary cirrhosis (PBC), non-alcoholic fatty liver disease (NAFLD), and non-alcoholic steatohepatitis (NASH).

Provided herein are methods of treating or preventing a liver disorder in a patient (e.g., a human patient) in need thereof with Compound 1, or a pharmaceutically acceptable salt thereof, wherein the liver disorder is selected from liver inflammation, liver fibrosis, alcohol induced fibrosis, primary sclerosing cholangitis (PSC), primary biliary cirrhosis (PBC), non-alcoholic fatty liver disease (NAFLD), and non-alcoholic steatohepatitis (NASH).

Also provided herein are methods of impeding or slowing the progression of non-alcoholic fatty liver disease (NAFLD) to non-alcoholic steatohepatitis (NASH) in a patient (e.g., a human patient) in need thereof comprising administering an SSAO inhibitor (such as Compound 1, or a pharmaceutically acceptable salt thereof. In some embodiments, the methods comprises administering a therapeutically effective amount of Compound 1. Also provided herein are methods of impeding or slowing the progression of NASH in a patient (e.g., a human patient) in need thereof comprising administering Compound 1, or a pharmaceutically acceptable salt thereof.

In some embodiments, provided herein are methods of treating a liver disorder in a patient in need thereof with Compound 1, or a pharmaceutically acceptable salt thereof, wherein the SSAO inhibitor selectively inhibits SSAO. Accordingly, in some embodiments, MAO-A (Monoamine oxidase A) is not inhibited. In some embodiments, MAO-B (Monoamine oxidase B) is not inhibited. In some embodiments MAO-A and MAO-B are not inhibited.

In some embodiments, the patient is a human. Obesity is highly correlated with NAFLD and NASH, but lean people can also be affected by NAFLD and NASH. Accordingly, in some embodiments, the patient is obese. In some embodiments, the patient is not obese. Obesity can be correlated with or cause other diseases as well, such as diabetes mellitus or cardiovascular disorders. Accordingly, in some embodiments, the patient also has diabetes mellitus and/or a cardiovascular disorder. Without being bound by theory, it is believed that comorbidities, such as obesity, diabetes mellitus, and cardiovascular disorders can make NAFLD and NASH more difficult to treat. Conversely, the only currently recognized method for addressing NAFLD and NASH is weight loss, which would likely have little to no effect on a lean patient.

The risk for NAFLD and NASH increases with age, but children can also suffer from NAFLD and NASH, with literature reporting of children as young as 2 years old (Schwimmer, et al., Pediatrics, 2006, 118:1388-1393). In some embodiments, the patient is 2-17 years old, such as 2-10, 2-6, 2-4, 4-15, 4-8, 6-15, 6-10, 8-17, 8-15, 8-12, 10-17, or 13-17 years old. In some embodiments, the patient is 18-64 years old, such as 18-55, 18-40, 18-30, 18-26, 18-21, 21-64, 21-55, 21-40, 21-30, 21-26, 26-64, 26-55, 26-40, 26-30, 30-64, 30-55, 30-40, 40-64, 40-55, or 55-64 years old. In some embodiments, the patient is 65 or more years old, such as 70 or more, 80 or more, or 90 or more.

NAFLD and NASH are common causes of liver transplantation, but patients that already received one liver transplant often develop NAFLD and/or NASH again. Accordingly, in some embodiments, the patient has had a liver transplant.

In some embodiments, the patient is at risk of developing an adverse effect prior to the administration in accordance with the methods provided herein. In some embodiments, the adverse effect is an adverse effect which affects the kidney, lung, heart, and/or skin.

In some embodiments, the patient has had one or more prior therapies. In some embodiments, the liver disorder progressed during the therapy.

In some embodiments, the methods do not comprise administering an antihistamine, an immunosuppressant, a steroid (such as a corticosteroid), rifampicin, an opioid antagonist, or a selective serotonin reuptake inhibitor (SSRI).

Also provided herein are dosing regimens for administering Compound 1, or a pharmaceutically acceptable salt thereof. In some embodiments, the therapeutically effective amount of Compound 1, or a pharmaceutically acceptable salt thereof, is a dose to achieve saturation of plasma SSAO. A dose that achieves saturation of plasma SSAO is a dose that reduces in plasma SSAO activity by at least about 80%, at least about 85%, at least about 90%, at least about 95%, at least about 98%, or at least about 99% relative to baseline levels. In some embodiments, the therapeutically effective amount of Compound 1, or a pharmaceutically acceptable salt thereof, is a dose that achieves saturation of liver SSAO. Administration of Compound 1, or a pharmaceutically salt thereof, at doses less than saturation of plasma SSAO are still efficacious. In some embodiments, Compound 1, or a pharmaceutically acceptable salt thereof, can be administered at a dose that achieves at least about 25% saturation of the plasma SSAO target. In other embodiments, Compound 1, or a pharmaceutically acceptable salt thereof, can be administered at a dose that achieves at least about 50% saturation of the plasma SSAO target. In other embodiments, Compound 1, or a pharmaceutically acceptable salt thereof, can be administered at a dose that achieves at least about 75% saturation of the plasma SSAO target. In other embodiments, Compound 1, or a pharmaceutically acceptable salt thereof, can be administered at a dose that achieves at least about 95% saturation of the plasma SSAO target. In other embodiments, Compound 1, or a pharmaceutically acceptable salt thereof, can be administered at a dose that achieves of about 100% saturation of the plasma SSAO target.

In some embodiments, Compound 1, or a pharmaceutically acceptable salt thereof, is administered daily to the individual at a dose to obtain a steady state plasma area under the curve from time 0 to infinity (AUC_{0-∞}) of from about 10 ng*h/mL to about 2,000 ng*h/mL. In some embodiments, the compound is administered daily to the individual at a dose that to obtain a steady state plasma area AUC_{0-∞} of from about 10 ng*h/mL to about 1,500 ng*h/mL. In some embodiments, the compound is administered daily to the individual at a dose that to obtain a steady state plasma area AUC_{0-∞} of from about 100 ng*h/mL to about 1,500 ng*h/mL. In some embodiments, the compound is administered daily to the individual at a dose that to obtain a steady state plasma area AUC_{0-∞} of from about 10 ng*h/mL to about 1.000 ng*h/mL. In some embodiments, the compound is administered daily to the individual at a dose that to obtain a steady state plasma area AUC_{0-∞} of from about 100 ng*h/mL to about 1,000 ng*h/mL. In some embodiments, the compound is administered daily to the individual at a dose that to obtain a steady state plasma area AUC_{0-∞} of from about 500 ng*h/mL to about 1,000 ng*h/mL. In some embodiments, the compound is administered daily to the individual at a dose that to obtain a steady state plasma area AUC_{0-∞} of from about 10 ng*h/mL to about 700 ng*h/mL. In some embodiments, the compound is administered daily to the individual at a dose that to obtain a steady state plasma area AUC_{0-∞} of from about 100 ng*h/mL to about 700 ng*h/mL. In some embodiments, the compound is administered daily to the individual at a dose that to obtain a steady state plasma area AUC_{0-∞} of from about 500 ng*h/mL to about 700 ng*h/mL. In some embodiments, the compound is administered daily to the individual at a dose that to obtain a steady state plasma area AUC_{0-∞} of from about 10 ng*h/mL to about 200 ng*h/mL. In some embodiments, the compound is administered daily to the individual at a dose that to obtain a steady state plasma area AUC_{0-∞} of from about 100 ng*h/mL to about 200 ng*h/mL. In some embodiments, the compound is administered daily to the individual at a dose that to obtain a steady state plasma area AUC_{0-∞} of from about 10 ng*h/mL to about 100 ng*h/mL. In some embodiments, the compound is administered daily to the individual at a dose that to obtain a steady state plasma area AUC_{0-∞} of from about 10 ng*h/mL to about 50 ng*h/mL. In some embodiments, the compound is administered daily to the individual at a dose that to obtain a steady state plasma area AUC_{0-∞} of from about 10 ng*h/mL to about 20 ng*h/mL.

In some embodiments of the disclosure, Compound 1, or a pharmaceutically acceptable salt thereof, can be administered to patients orally once daily at doses as low as 1 mg or less and still sufficiently reduce amine oxidase activity and reduce lymphocyte adhesion and transmigration. For example, Compound 1, or a pharmaceutically acceptable salt thereof, can be administered orally once daily to a patient with a liver disorder (e.g. NASH) at a dose of from about 0.5 mg to about 25 mg. In some embodiments, Compound 1, or a pharmaceutically acceptable salt thereof, can be administered orally once daily to a patient with a liver disorder (e.g. NASH) at a dose of from about 1 mg to about 15 mg. In some embodiments, Compound 1, or a pharmaceutically acceptable salt thereof, can be administered orally once daily to a patient with a liver disorder (e.g. NASH) at a dose of from about 2 mg to about 10 mg. In some embodiments, Compound 1, or a pharmaceutically acceptable salt thereof, can be administered orally once daily to a patient with a liver disorder (e.g. NASH) at a dose of about 1 mg. In some embodiments, Compound 1, or a pharmaceutically acceptable salt thereof, can be administered orally once daily to a patient with a liver disorder (e.g. NASH) at a dose of about 4 mg. In some embodiments, Compound 1, or a pharmaceutically acceptable salt thereof, can be administered orally once daily to a patient with a liver disorder (e.g. NASH) at a dose of about 5 mg. In some embodiments, Compound 1, or a pharmaceutically acceptable salt thereof, can be administered orally once daily to a patient with a liver disorder (e.g. NASH) at a dose of about 10 mg.

In some embodiments, from about 0.5 mg to about 50 mg of Compound 1, or a pharmaceutically acceptable salt thereof, is administered to the individual. In particular embodiments, the compound is administered once daily orally to the patient. In other embodiments, the compound is administered once every other day to the patient. In other embodiments, the compound is administered twice weekly to the patient. In other embodiments, the compound is administered once weekly to the patient. In some embodiments, about 1 mg to about 5 mg of the compound is administered to the individual. In some embodiments about 1 mg to about 3 mg of the compound is administered to the individual. In some embodiments about 5 mg to about 10 mg of the compound is administered to the individual. In some embodiments, about 10 mg to about 15 mg of the compound is administered to the individual. In some embodiments, about 15 mg to about 20 mg of the compound is administered to the individual. In some embodiments, about 20 mg to about 25 mg of the compound is administered to the individual. In some embodiments, about 25 mg to about 30 mg of the compound is administered to the individual. In some embodiments, about 30 mg to about 35 mg of the compound is administered to the individual. In some embodiments, about 35 mg to about 40 mg of the compound is administered to the individual. In some embodiments, about 40 mg to about 45 mg of the compound is administered to the individual. In some embodiments, about 45 mg to about 50 mg of the compound is administered to the individual. In some embodiments, about 0.5 mg of the compound is administered to the individual. In some embodiments, about 1 mg of the compound is administered to the individual. In some embodiments, about 2 mg of the compound is administered to the individual. In some embodiments, about 3 mg of the compound is administered to the individual. In some embodiments, about 4 mg of the compound is administered to the individual. In some embodiments, about 5 mg of the compound is administered to the individual. In some embodiments, about 6 mg of the compound is administered to the individual. In some embodiments, about 7 mg of the compound is administered to the individual. In some embodiments, about 8 mg of the compound is administered to the individual. In some embodiments, about 9 mg of the compound is administered to the individual. In some embodiments, about 10 mg of the compound is administered to the individual. In some embodiments, about 15 mg of the compound is administered to the individual. In some embodiments, about 20 mg of the compound is administered to the individual. In some embodiments, about 25 mg of the compound is administered to the individual. In some embodiments, about 30 mg of the compound is administered to the individual. In some embodiments, about 35 mg of the compound is administered to the individual. In some embodiments, about 40 mg of the compound is administered to the individual. In some embodiments, about 45 mg of the compound is administered to the individual. In some embodiments, about 50 mg of the compound is administered to the individual.

The treatment period generally can be one or more weeks. In some embodiments, the treatment period is at least 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more. In some embodiments, the treatment period is from about a week to about a month, from about a month to about a year, from about a year to about several years. In some embodiments, the treatment period at least any of about 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more. In some embodiments, the treatment period is the remaining lifespan of the patient.

In some embodiments, the amount of Compound 1, or a pharmaceutically acceptable salt thereof, is administered on day 1 of the treatment period are greater than or equal to the amounts administered on all subsequent days of the treatment period. In some embodiments, the amounts administered on day 1 of the treatment period are equal to the amounts administered on all subsequent days of the treatment period.

Compound 1, or a pharmaceutically acceptable salt thereof, used in accordance with the method described herein can be administered to an individual a once daily dose for a first period of time, followed by a second period of time in which administration of the compound is discontinued, wherein the SSAO inhibitory activity is maintained during both the first and the second period of time. In some embodiments, the first and second periods of time are each one-week periods. For example, provided herein is a method of treatment in an individual for a period of 14 days comprising administering to the individual a once daily dose of Compound 1, or a pharmaceutically acceptable salt thereof, for a first 7 days, followed by discontinued administration of the compound for the following 7 days, wherein the SSAO inhibitory activity is maintained in the individual during the entire 14-day period. As another example, provided herein is a method of treatment in an individual for a period of four weeks, comprising administering to the individual a once daily dose of Compound 1, or a pharmaceutically acceptable salt thereof, for a first two weeks, followed by discontinued administration of the compound for the following two weeks, wherein the SSAO inhibitory activity is maintained in the individual during the entire four-week period. In some embodiments, the daily dose is about 1 mg, about 3 mg, about 4 mg, about 5 mg, about 6 mg, about 10 mg, about 15 mg, about 20 mg, about 25 mg, about 30 mg, or about 40 mg.

In some embodiments, administration of Compound 1, or a pharmaceutically acceptable salt thereof, decreases liver inflammation in the individual. Methods of assessing liver inflammation are known to the skilled artisan and may include histological analysis and assignment of histological score of lobular inflammation. Thus it is understood that methods of treatment detailed herein, in some embodiments, comprise treating a liver disorder such as liver inflammation, liver fibrosis, alcohol induced fibrosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing lobular inflammation or histological markers associated with lobular inflammation.

In some embodiments, Compound 1, or a pharmaceutically acceptable salt thereof, decreases liver fibrosis in the individual. Methods of assessing liver fibrosis are known to the skilled artisan and may include histological analysis. Thus it is understood that methods of treatment detailed herein, in some embodiments, comprise treating a liver disorder such as liver inflammation, liver fibrosis, alcohol induced fibrosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing fibrosis or histological markers associated with fibrosis.

In some embodiments, administration of Compound 1, or a pharmaceutically acceptable salt thereof, decreases at least one of liver inflammation and fibrosis in the individual. In some embodiments, administration decreases liver inflammation and fibrosis in the individual. Thus it is understood that methods of treatment detailed herein, in some embodiments, comprise treating a liver disorder such as liver inflammation, liver fibrosis, alcohol induced fibrosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing at least one of lobular inflammation, fibrosis, or histological markers of any of the foregoing.

In some embodiments, administration Compound 1, or a pharmaceutically acceptable salt thereof, decreases serum alanine aminotransferase in the individual. Thus it is understood that methods of treatment detailed herein, in some embodiments, comprise treating a liver disorder such as liver inflammation, liver fibrosis, alcohol induced fibrosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises reducing serum alanine aminotransferase.

Also provided herein Compound 1, or a pharmaceutically acceptable salt thereof, for use in treating a liver disorder such as liver inflammation, liver fibrosis, alcohol induced fibrosis, primary sclerosing cholangitis (PSC), primary biliary cirrhosis (PBC), non-alcoholic fatty liver disease (NAFLD), and non-alcoholic steatohepatitis (NASH) an individual in need thereof, using the methods as described herein.

Also provided herein are uses of the Compound 1, or a pharmaceutically acceptable salt thereof, for manufacture of a medicament for treating a liver disorder such as liver inflammation, liver fibrosis, alcohol induced fibrosis, primary sclerosing cholangitis (PSC), primary biliary cirrhosis (PBC), non-alcoholic fatty liver disease (NAFLD), and non-alcoholic steatohepatitis (NASH) an individual in need thereof, using the methods as described herein.

### Combinations

The present disclosure further provides combinations of Compound 1, or a pharmaceutically acceptable salt thereof, with other therapeutic agents that are used to treat liver diseases. In particular, the present disclosure provides for combinations of Compound 1, or a pharmaceutically acceptable salt thereof, and other therapeutic agents used in the treatment of NASH. Owing to its low clinical dose, as disclosed herein, Compound 1, or a pharmaceutically acceptable salt thereof, is an attractive candidate for use in fixed-dose combinations for the treatment of NASH.

In some embodiments, the Compound 1, or a pharmaceutically acceptable salt thereof, is administered in combination with a Farnesoid X Receptor (FXR) agonist. In some embodiments, the FXR agonist is obeticholic acid. In some embodiments, the FXR agonist is cilofexor. In some embodiments, the FXR agonist is tropifexor. In some embodiments, the FXR agonist is EYP001 (Vonafexor, proposed INN). In some embodiments, the FXR agonist is MET409 (Metacrine). In some embodiments, the FXR agonist is EDP-305 (by Enanta). In some embodiments, the FXR agonist is

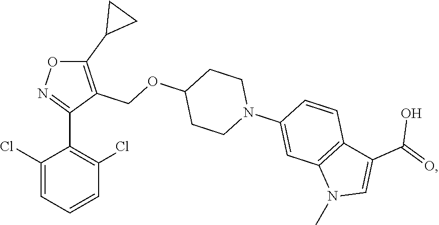 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof.

In some embodiments, the Compound 1, or a pharmaceutically acceptable salt thereof, is administered in combination with a peroxisome proliferator-activated receptor (PPAR) agonist. In some embodiments, the PPAR agonist is pioglitazone. In some embodiments, the PPAR agonist is rosiglitazone. In some embodiments, the PPAR agonist is elafibranor. In some embodiments, the PPAR agonist is saroglitazar. In some embodiments, the PPAR agonist is lanifibranor. In some embodiments, the PPAR agonist is seladelpar.

In some embodiments, the Compound 1, or a pharmaceutically acceptable salt thereof, is administered in combination with a pan-caspase inhibitor. In some embodiments, the pan-caspase inhibitor is emricasan.

In some embodiments, the Compound 1, or a pharmaceutically acceptable salt thereof, is administered in combination with a galectin-3 inhibitor. In some embodiments, the galectin-3 inhibitor is belapectin.

In some embodiments, the Compound 1, or a pharmaceutically acceptable salt thereof, is administered in combination with a stearoyl Co-A desaturase 1 inhibitor. In some embodiments, the stearoyl Co-A desaturase 1 inhibitor is armachol.

In some embodiments, the Compound 1, or a pharmaceutically acceptable salt thereof, is administered in combination with a chemokine receptor type 2 and 5 (CCR2/CCR5 chemokine) antagonist. In some embodiments, the CCR2/CCR5 chemokine agonist is cenicriviroc.

In some embodiments, the Compound 1, or a pharmaceutically acceptable salt thereof, is administered in combination with an antioxidant. In some embodiments, the antioxidant is Vitamin E.

### Articles of Manufacture and Kits

The present disclosure further provides articles of manufacture comprising a compound described herein, or a salt thereof, a composition described herein, or one or more unit dosages described herein in suitable packaging. In certain embodiments, the article of manufacture is for use in any of the methods described herein. Suitable packaging (e.g., containers) is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises at least two compounds described herein, or a pharmaceutically acceptable salt thereof, or a composition comprising a compound described herein, or a pharmaceutically acceptable salt thereof. The kits may employ any of the compounds disclosed herein or a pharmaceutically acceptable salt thereof. In some embodiments, the kit employs Compound 1, or a pharmaceutically acceptable salt thereof, as described herein. The kits may be used for any one or more of the uses described herein, and, accordingly, may contain instructions for the treatment as described herein.

Kits generally comprise suitable packaging. The kits may comprise one or more containers comprising any compound described herein or a pharmaceutically acceptable salt thereof. Each component can be packaged in separate containers or some components can be combined in one container where cross-reactivity and shelf life permit. In some embodiments, the kit includes a container comprising Compound 1, or a pharmaceutically acceptable salt thereof,

The kits may be in unit dosage forms, bulk packages (e.g., multi-dose packages) or sub-unit doses. For example, kits may be provided that contain sufficient dosages of a compound as disclosed herein, or a pharmaceutically acceptable salt thereof, and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### EXAMPLES

The treatment provided herein can be tested by administering agent or agents to a well-known mouse model and evaluating the results. Methods of such testing can be adapted from those known. See, e.g., US Pat. Pub. No. 2015/0342943, incorporated herein by reference.

### Example 1

### Background

Semicarbazide-sensitive amine oxidase (SSAO) contributes to non-alcoholic steatohepatitis (NASH) by increasing oxidative stress through deamination of primary amines (e.g., methylamine, MMA) to aldehyde, ammonium, and H₂O₂ and by recruitment of inflammatory cells to the liver, exacerbating hepatic inflammation and injury. SSAO levels are elevated in NASH and correlate with fibrosis stage. Compound 1 is a selective, covalent SSAO inhibitor that decreases liver inflammation and fibrosis in a rat model of NASH. A single-ascending dose clinical trial of Compound 1 was performed.

### Methods

Four groups of 8 healthy participants were randomized to receive Compound 1 capsule or matching placebo in a 3:1 ratio. Plasma levels of Compound 1 and PD biomarkers were determined at pre-dose and various time points post-dose. SSAO inhibition was determined by measuring relative reductions in plasma H₂O₂ generation after addition of an exogenous substrate (benzylamine). Endogenous methylamine (MMA) levels, predicted to increase upon SSAO inhibition, were measured in plasma. Safety was assessed for 7 (±3) days after dosing.

Plasma samples for Compound 1 concentration and SSAO activity determination were collected at 0.25, 0.5, 1, 2, 3, 4, 6, 8, 10, 12, 24, 48 (SSAO activity only), and 168 (SSAO activity only) hours after administration of a single dose of study medication (placebo or compound). Plasma PK parameters were determined by non-compartmental analysis. SSAO activity was assessed by measuring hydrogen peroxide (H₂O₂) generation levels in plasma samples from placebo and active Compound 1 recipients. Percent change in total amine oxidase activity was determined relative to the corresponding pre-dose (baseline) samples.

SSAO-specific amine oxidase levels in plasma were determined using a kinetic-based assay essentially as described previously (Schilter et al). Endogenous monoamine oxidases A and B were inhibited by adding pargyline to plasma samples prior to measuring H₂O₂ generation levels in placebo and active recipients. Maximum inhibition was defined by pre-dose (baseline) samples additionally treated with a high dose of Compound 1 and percent changes in SSAO-specific activity were calculated relative to baseline samples.

### Results

32 healthy human participants (100% male, 63% Black, 19% Asian, 13% Caucasian) were enrolled and received a single oral dose of Compound 1 (1, 3, 6, and 10 mg, n=6 each) or placebo (n=2). Compound 1 plasma PK exposure increased in a greater than dose proportional manner between the 3 and 10 mg dose levels. The mean half-life of Compound 1 ranged from 1-3 hours. At 4 hours post-dose, near complete inhibition of plasma SSAO activity was seen in all dose cohorts and continued suppression was detected for up to 1 week after a single dose of Compound 1. Maximum plasma MMA levels increased with Compound 1. No clinically relevant adverse events or laboratory abnormalities were reported.

As shown in Table 1, doses 1, 3, 6, and 10 mg of Compound 1 were all well tolerated.

TABLE 1
Compound 1 Treatment Associated Adverse Events

| | 1 mg of a | 3 mg of a | 6 mg of a | 10 mg of a | |
|---|---|---|---|---|---|
| | tosylate | tosylate | tosylate | tosylate | |
| | salt of | salt of | salt of | salt of | |
| | Compound | Compound | Compound | Compound | |
| | 1 or | 1 or | 1 or | 1 or | |
| | placebo | placebo | placebo | placebo | All |
| | (n = 8) | (n = 8) | (n = 8) | (n = 8) | (n = 32) |
| Subject | 0 | 0 | 2 (25) | 3 (37.5) | 5 (15.6) |
| incidence | | | | | |
| of any | | | | | |
| TEAE | | | | | |
| Subject | 0 | 0 | 0 | 1 (12.5) | 1 (3.1) |
| incidence | | | | | |
| of TEAEs | | | | | |
| considered | | | | | |
| possibly | | | | | |
| treatment- | | | | | |
| related | | | | | |
| TEAE | | | | | |
| diagnosis | | | | | |
| and | | | | | |
| frequency | | | | | |
| constipation | 0 | 0 | 0 | 1 (12.5) | 1 (3.1) |
| contact | 0 | 0 | 2 (25) | 0 | 2 (6.3) |
| dermatitis | | | | | |
| dysgeusia | 0 | 0 | 0 | 1 (12.5) | 1 (3.1) |
| headache | 0 | 0 | 0 | 1 (12.5) | 1 (3.1) |
| oral herpes | 0 | 0 | 0 | 1 (12.5) | 1 (3.1) |
| sore throat | 0 | 0 | 1 (12.5) | 0 | 1 (3.1) |
| upper | 0 | 0 | 0 | 1 (12.5) | 1 (3.1) |
| respiratory | | | | | |
| tract | | | | | |
| infection | | | | | |

Single doses of the tosylate salt of Compound 1 were rapidly cleared from plasma and exhibited greater than dose proportional plasma PK between 3 and 10 mg.

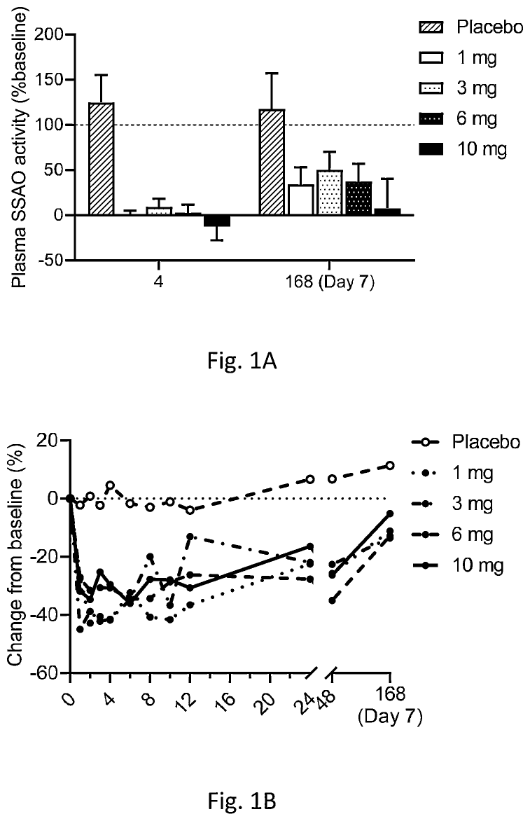

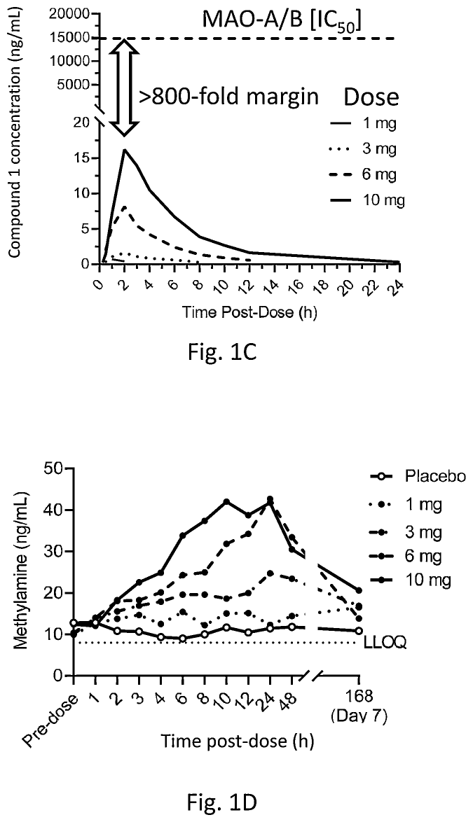

Single doses of Compound 1 rapidly and potently decreased plasma amine oxidase activity in all subjects as shown in FIG. 1A and FIG. 1B. Near complete inhibition of SSAO-specific activity as observed at 4 hours post dose (FIG. 1A and FIG. 1B). Inhibition of plasma SSAO amine oxidase activity and dose-dependent increases in plasma MMA were sustained up to 1 week after single doses of Compound 1, suggesting potent, covalent target engagement and supporting once daily dosing despite a short plasma half-life (FIG. 1A and FIG. 1B).

The concentrations (Cₘₐₓ) for Compound 1 for SSAO were more than 800 times lower than the IC₅₀ concentrations for MAO-A and MAO-B at all dose levels (FIG. 1C, Table 2).

TABLE 2
Biochemical activity (IC₅₀ μM)

| | SSAO inhibitor | | |
|---|---|---|---|
| | SSAO | MAO-A | MAO-B |
| Compound 1 | 0.0065 | >50 | >50 |
| BI 1467335 | 0.005 | >100 | 2.7 |
| (PXS-4728A) | | | |

Dose-dependent increases in methylamine were observed, indicating potent plasma SSAO target engagement across the dose range. FIG. 1D.

### Conclusions

Compound 1 was safe and well tolerated in healthy subjects administered a single oral dose ranging from 1 mg to 10 mg. Compound 1 inhibited SSAO activity for up to seven days after a single dose. This suggests that Compound 1 may be effective for treating liver diseases or disorders by selectively inhibiting SSAO. It may also exhibit SSAO activity for seven days after only a single dose, suggesting that daily administration for one week may exert a therapeutic effect for a two-week period.

### Example 2

A multiple-ascending dose clinical trial of Compound 1 was performed. 3 groups of 8 healthy participants were randomized to receive multiple once daily (QD) doses of Compound 1 or matching placebo in a 3:1 ratio for 7 days (1 mg and 4 mg) or 14 days (10 mg). Plasma levels of Compound 1 and PD biomarkers (plasma amine oxidase activity and methylamine levels) were determined at pre-dose and various timepoints post-dose. Safety, including laboratory, vital signs, and ECG, among others, was assessed for up to 14 days after last dose with no notable findings across subjects. All adverse events were considered mild (grade 1), except for one moderate (grade 2) adverse event in the placebo treatment group. No subject discontinued due to an adverse event (see Table 3).

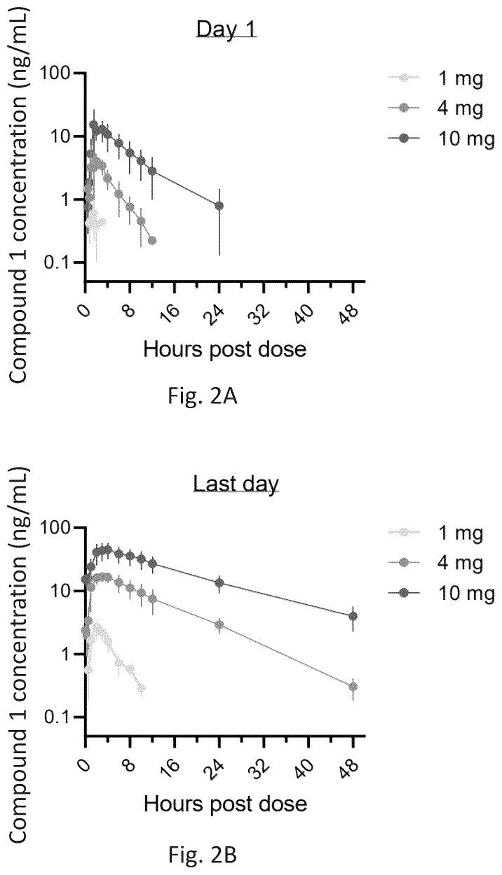

Compound 1 plasma PK exposure increases were greater than dose proportional between dose groups on Day 1, and significant accumulation at each dose level was observed after multiple QD doses. The accumulation ratio between the first and last day of dosing decreased as dose increased. Steady state was achieved in the highest dose cohort (10 mg) after 7 days. Compound 1 half-life increased with dose, consistent with a saturable target-mediated clearance (see FIG. 2A and FIG. 2B, Table 4).

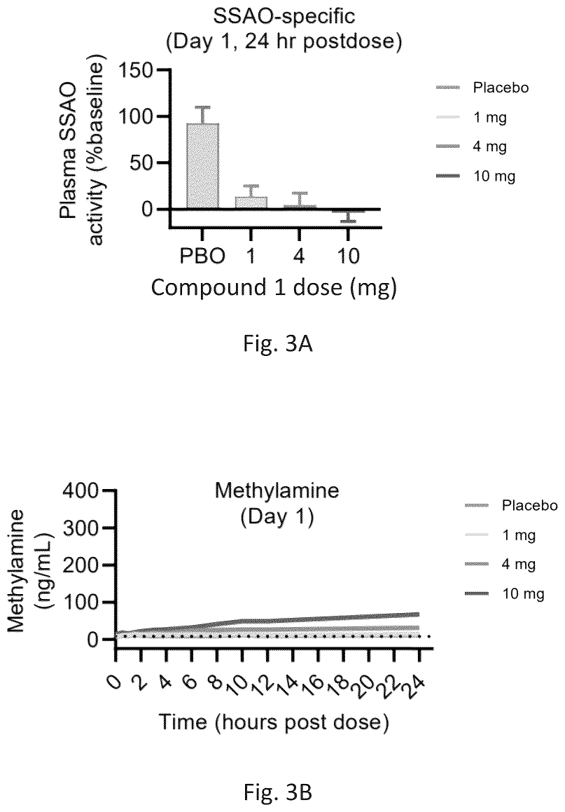

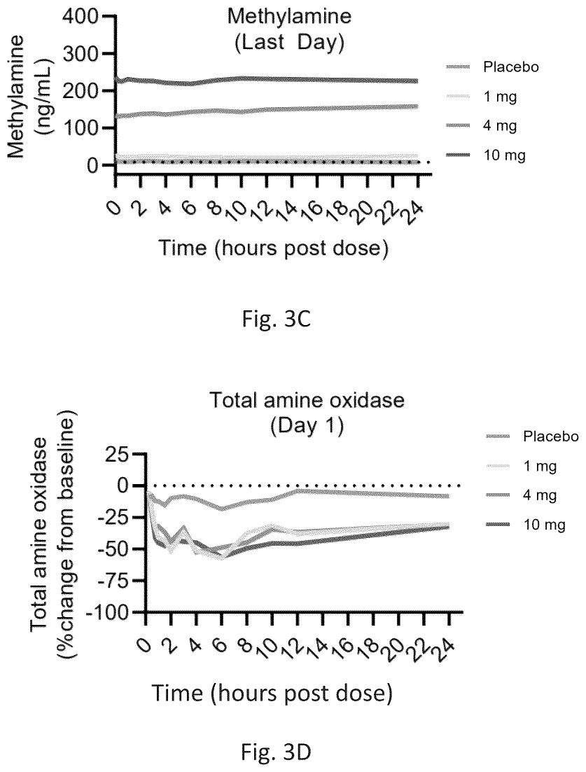

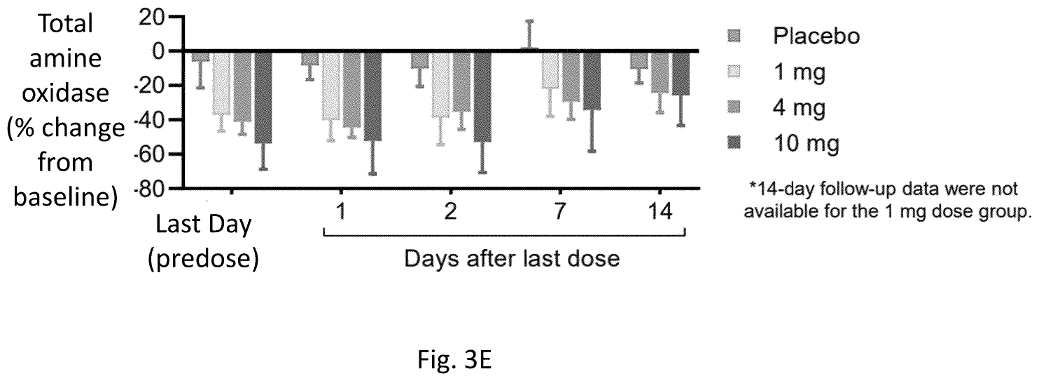

Near complete inhibition of plasma SSAO activity was seen on Day 1 in all dose cohorts (FIG. 3A), resulting in an increase in methylamine (FIG. 3B), which is an endogenous substrate of SSAO. Plasma methylamine levels increased in a dose proportional manner (FIG. 3B). After multiple doses, further increases in plasma methylamine were observed on the last day of Compound 1 administration (FIG. 3C). Inhibition of total amine oxidase was incomplete due to the presence of plasma amine oxidases that were not inhibited by Compound 1 (e.g., MAO-A, MAO-B) (FIG. 3D, E)

Compound 1 was safe and well tolerated in healthy subjects when administered up to 10 mg QD for 14 days. Steady state levels of Compound 1 were achieved after 7 days of dosing supporting a QD dosing regimen. Near complete inhibition of plasma SSAO amine oxidase activity and dose-dependent increases in plasma methylamine were sustained up to 2 weeks after cessation of dosing, suggesting that daily administration of Compound 1 for two weeks may exert a therapeutic effect for a two-week period after cessation of dosing.

TABLE 3
Compound 1 Treatment Associated Adverse Events

| | | 1 mg | 4 mg | 10 mg | Overall |
|---|---|---|---|---|---|
| | | Compound | Compound | Compound | Compound |
| | Placebo | 1 | 1 | 1 | 1 |
| | (n = 6) | (n = 6) | (n = 6) | (n = 6) | (n = 18) |
| Subject | 3 (50) | 2 (33.3) | 2 (33.3) | 6 (100) | 10 (55.6) |
| incidence of | | | | | |
| any TEAE, n | | | | | |
| (%) | | | | | |
| Subject | 1 (16.7) | 1 (16.7) | 0 | 0 | 1 (5.6) |
| incidence of | | | | | |
| TEAEs | | | | | |
| considered | | | | | |
| possibly | | | | | |
| treatment- | | | | | |
| related¹, n | | | | | |
| (%) | | | | | |

TEAE diagnosis and frequency

| | | | | | |
|---|---|---|---|---|---|
| Back pain | 1 (16.7) | 0 | 0 | 0 | 0 |
| Catheter site | 1 (16.7) | 0 | 0 | 0 | 0 |
| inflammation | | | | | |
| Contusion | 1 (16.7) | 0 | 0 | 0 | 0 |
| Dermatitis | 0 | 1 (16.7) | 2 (33.3) | 0 | 3 (16.7) |
| contact | | | | | |
| Diarrhea | 1 (16.7) | 0 | 0 | 0 | 0 |
| Dizziness | 0 | 1 (16.7) | 0 | 0 | 1 (5.6) |
| Headache | 0 | 1 (12.5) | 0 | 0 | 1 (5.6) |
| Medical | 2 (33.3) | 0 | 0 | 6 (100) | 6 (33.3) |
| device site | | | | | |
| reaction² | | | | | |
| Rhinitis | 0 | 0 | 0 | 1 (16.7) | 1 (5.6) |

¹One subject who received 1 mg Compound 1 for 7 days had an event (headache) considered possibly related to treatment.
²All 8 subjects (6 Compound 1, 2 placebo) in the 10 mg cohort had mild events of contact dermatitis at the site of ECG leads (“Medical device site reaction”); ECGs were at least daily, per protocol.

TABLE 4
Compound 1 Pharmacokinetic data

| Dose | | Cₘₐₓ | Tₘₐₓ | AUC₀₋ₜ | t_{1/2} | AR | AR |
|---|---|---|---|---|---|---|---|
| (mg) | Day | (ng/mL) | (h) | (h*ng/mL) | (h) | (Cₘₐₓ) | (AUC) |
| 1 | 1 | 0.568 (64) | 1.53 (0.82, 2.00) | NC | | | |
| | Last | 2.75 (36) | 2.51 (1.02, 3.00) | 13.3 (14) | 2.27 (2.00, 2.48) | 5.98 (49) | NC |
| 4 | 1 | 5.11 (64) | 2.02 (1.02, 3.07) | 17.1 (29) | | | |
| | Last | 18.6 (20) | 3.03 (1.03, 3.07) | 198 (32) | 6.71 (2.85, 8.13 | 4.32 (34) | 11.7 (22) |
| 10 | 1 | 17.7 (49) | 1.90 (1.48, 4.00) | 102 (53) | | | |
| | Last | 47.5 (31) | 3.98 (2.03, 4.02) | 656 (29) | 13.3 (9.97, 14.9) | 3.35 (57) | 8.2 (63) |

Tₘₐₓ and t_{1/2} presented as: median (min, max).
All other PK parameters presented as: mean (CV %).
AR = accumulation ratio between Day 1 and Last Day.
NC = not calculable.

### Example 3

A Multi-Center, Randomized, Double-Blind, Dose-Ranging, Placebo-Controlled, Clinical Study to Evaluate the Safety, Pharmacokinetics, Pharmacodynamics, and Preliminary Efficacy of Orally Administered Compound 1 in Patients with Presumed Non-Cirrhotic Non-Alcoholic Steatohepatitis (NASH) is performed. The total duration of study participation is approximately 22 weeks, consisting of a 6-week Screening Period, a 12-week Treatment Period and a 4-week Follow-up Period.

Approximately 80 clinically or histologically diagnosed adult non-cirrhotic presumed NASH patients who meet study eligibility criteria are enrolled and randomized at an overall ratio of 2:1 into 3 dose groups and placebo across 2 parts of the study. Eligibility criteria include: overweight or obese, with NASH by clinical diagnosis or biopsy, ALT≥43 U/L (men) or ≥28 U/L (women), MRI-cT1 liver inflammation >800 ms.

Part 1 of this study assesses 10 mg of Compound 1, the highest dose studied in Example 2. In Example 2, this dose led to >90% suppression of plasma VAP-1/SSAO-specific amine oxidase activity in healthy participants. NASH patients are expected to have a higher baseline level of VAP-1, and thus the PD effect of Compound 1 in NASH patients may differ from healthy participants. A higher dose of up to 20 mg may be enrolled based on assessment of safety and PK with the 10 mg dose, and a lower dose of 4 mg may be enrolled based on observation of a robust PD effect of Compound 1 on plasma VAP-1/SSAO activity in the 10 mg cohort, thus minimizing the number of patients exposed until the PK and PD effects of Compound 1 can be confirmed in Part 1 of the study.

In Part 1, approximately 30 patients will receive 10 mg Compound 1 (n=20) or matching placebo (n=10) orally once daily, for 12 weeks. Approximately 12 randomized patients take part in an intensive PK and PD collection after the first dose (Week 0/Day 1), at Week 6, and after the last dose of study drug (Week 12). Randomization will ensure approximately 8 patients in the Compound 1 group and approximately 4 patients in the placebo group are assigned to the PK/PD sub-study. Patients who are not participating in the PK/PD sub-study will have trough PK/PD sampling only.

An interim analysis is performed once all patients in Part 1 have completed Week 6 assessments. Interim PK and PD data are assessed. Blinded safety data is also reviewed. If robust VAP-1/SSAO activity suppression is observed and available PK from Part 1 suggests that a lower dose may also possibly lead to robust VAP-1/SSAO activity suppression, enrollment in Part 2 with 4 mg Compound 1 may be initiated. If safety data indicates 10 mg Compound 1 is overall safe and well-tolerated, and available PK from Part 1 predicts that targeted exposures for a dose up to 20 mg will be below an upper limit of 5400 ng·hr/mL for AUC₀₋₂₄ₕᵣ and 768 ng/ml for Cₘₐₓ, enrollment in Part 2 with a dose up to 20 mg Compound 1 may be initiated.

In Part 2, approximately 50 patients receive 4 mg Compound 1 (n=20), and/or up to 20 mg Compound 1 (n=20), or matching placebo (n=10) orally once daily, for 12 weeks. Approximately 15 randomized patients take part in an intensive PK and PD collection after the first dose (Week 0/Day 1), at Week 6, and after the last dose of study drug (Week 12). Randomization will ensure approximately 6 patients at each Compound 1 dose level and approximately 3 patients in the placebo group are assigned to the PK/PD sub-study. Patients who are not participating in the PK/PD sub-study will have trough PK/PD sampling only.

### PK/PD Sub-Study and Trough PK/PD Sampling

Plasma samples are collected for measurement of plasma concentrations of study drug and metabolites. Urine samples are collected for measurement of urine concentrations of study drug and metabolites. Patients who are not participating in the PK/PD sub-study undergo trough PK sampling only. Blood collection for NASH/fibrosis markers (CK-18 (M30 and M65), PIIINP, TIMP-1, HA, PRO-C3, and C3M) and inflammation markers (hs-CRP, IL-6, ICAM-1, and VCAM-1) are also conducted. The following exploratory fibrosis scores may also be calculated: FIB-4, enhanced liver fibrosis (ELF), and NAFLD. The PRO-C3/C3M ratio may be calculated. Patients are monitored for adverse events.

Plasma and urine samples are collected pre-dose for all patients on days 1, 2, 15, 29, 43, 57, and 85. For patients within the PK/PD sub-study: On days 1 (week 0), 43 (week 6), and 85 (week 12), plasma samples are collected at 30 minutes, 1 h, 2 h, 4 h, 6 h, and 8 h post-dose, and total urine collection is performed from 0-8 h post-dose. On days 2 (week 0), 44 (week 6), and 96 (week 12), samples are collected (24 h post-dose). On days 87 and 88, samples are collected (48 and 72 hours post-week 12 dose). Markers including ALT, AST, ALP, and total bilirubin will be monitored.

### Example 4

### Methods

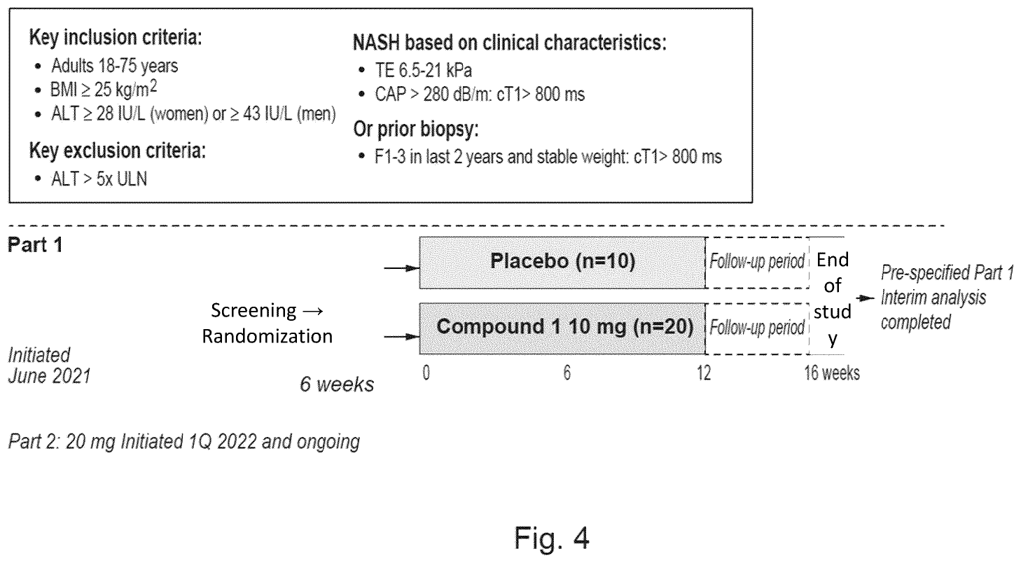

Part 1 of the two-part trial was a double-blind, placebo-controlled study in 30 adults with non-cirrhotic NASH phenotype evaluating 10 mg Compound 1 once daily (QD) for 12 weeks followed by off-treatment evaluation at week 16 (FIG. 4). Part 1 interim analysis primary endpoint was safety assessed by adverse events (AEs) and laboratory tests; percent change from baseline (BL) in plasma VAP-1 activity was a secondary endpoint. Exploratory imaging and blood-based biomarkers of liver inflammation and fibrosis were also assessed. Analyses of change (or percent change) from baseline used an ANCOVA model with change (or percent change) from baseline as the dependent variable including treatment group and randomization strata as fixed effects and baseline as a covariate.

### Results

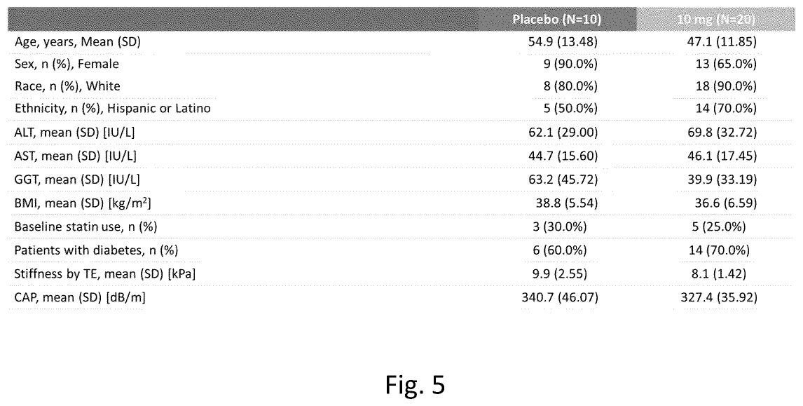

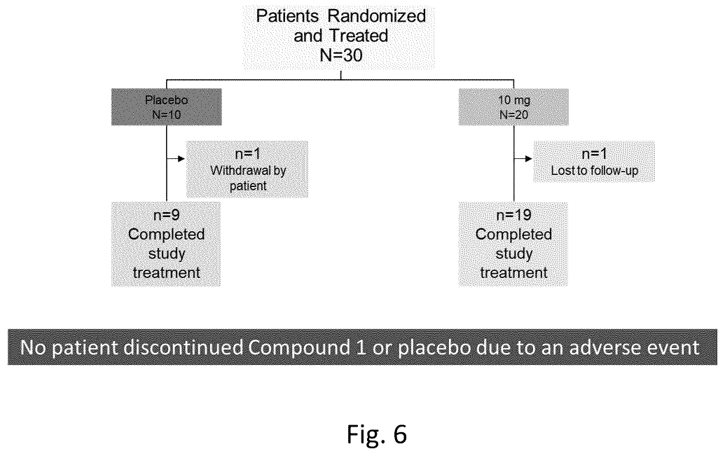

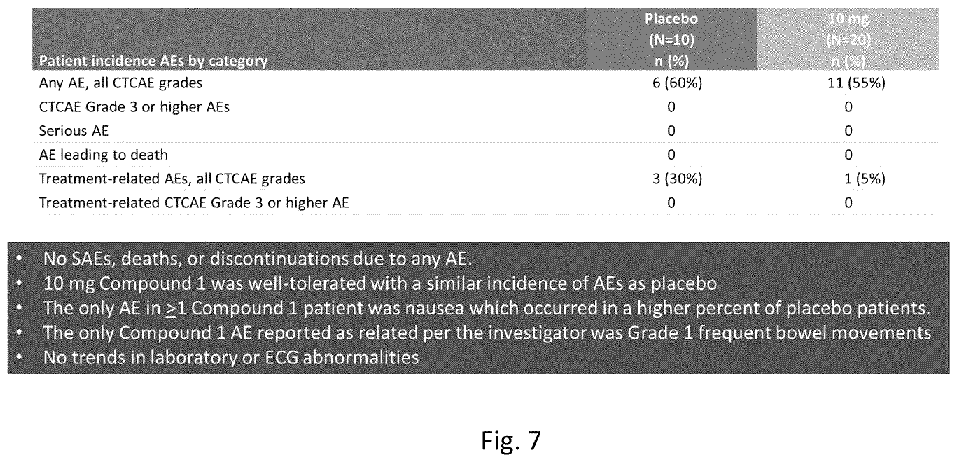

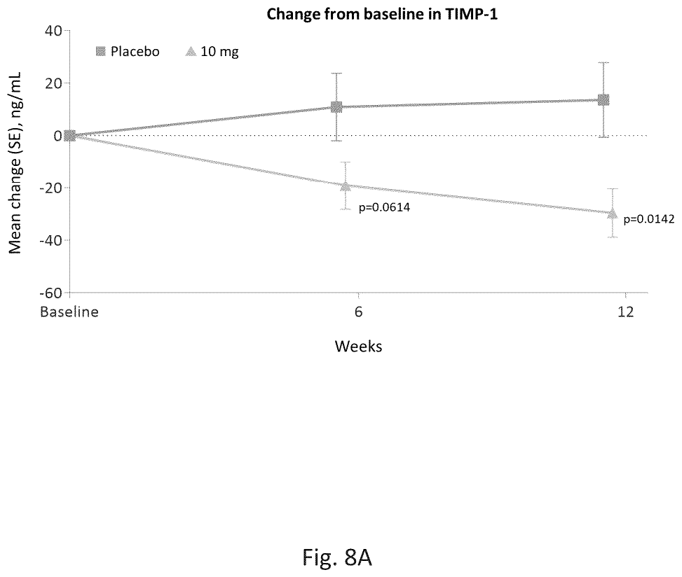

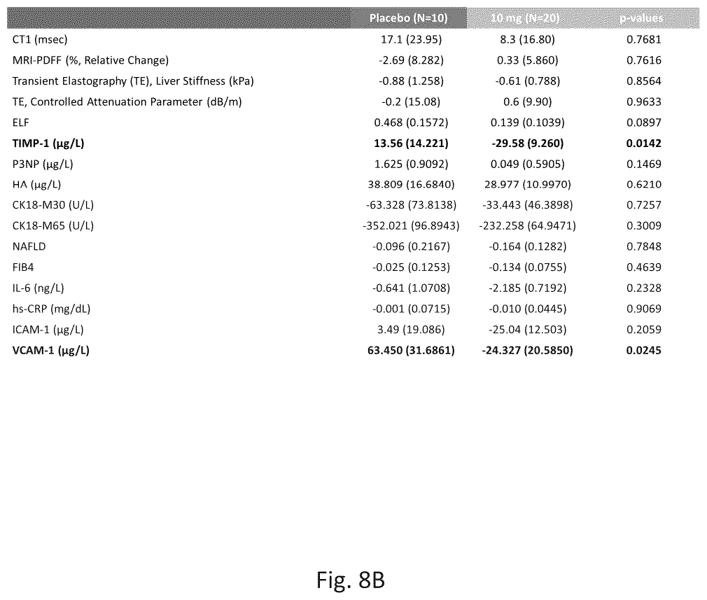

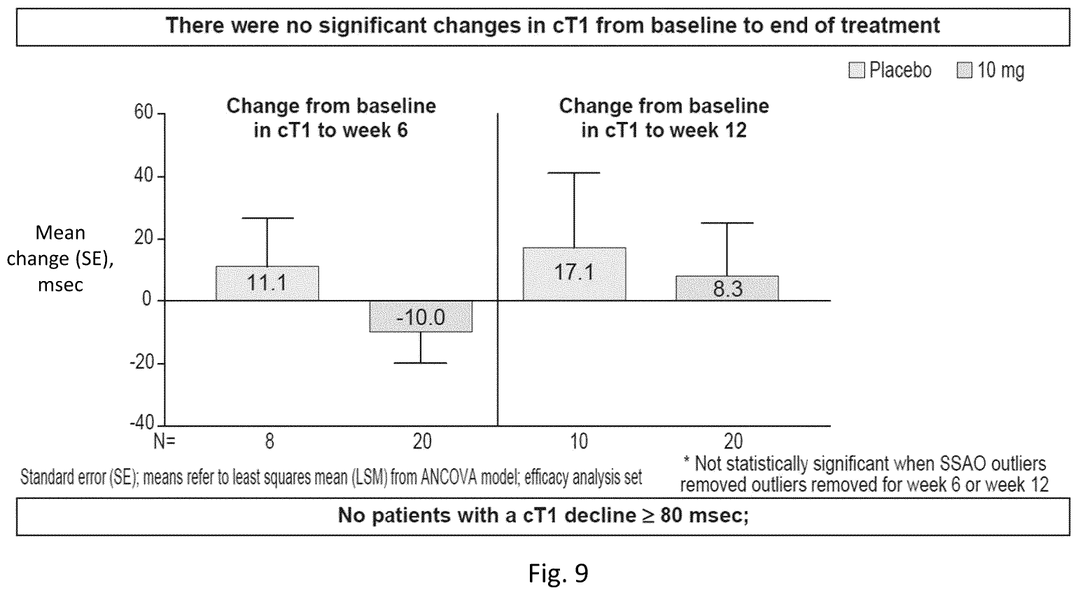

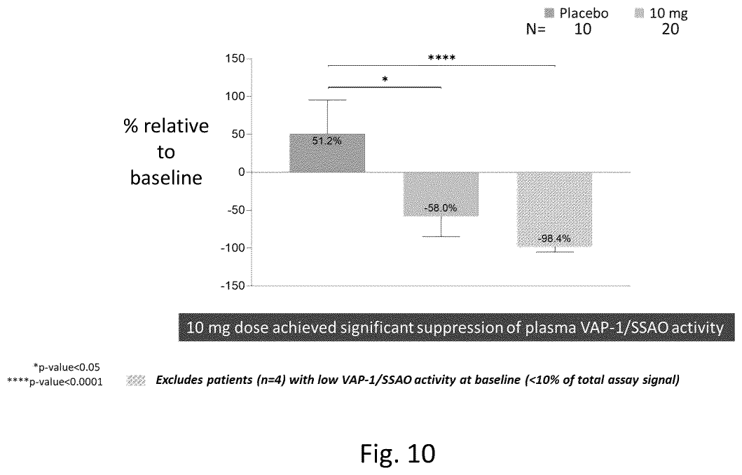

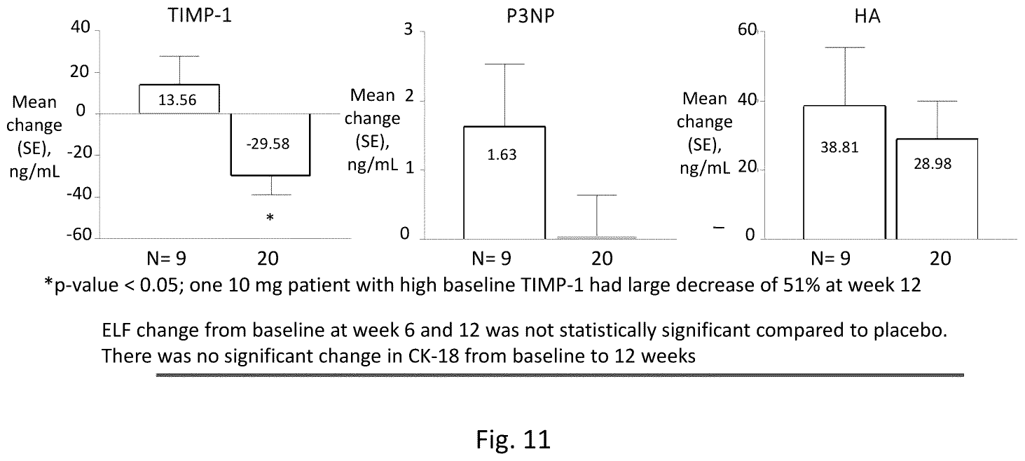

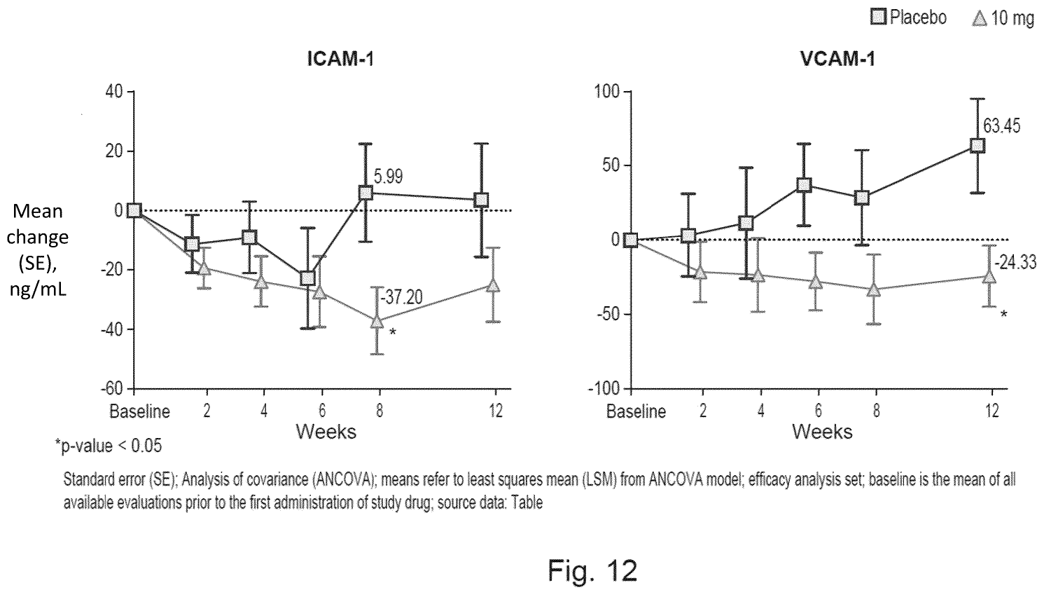

The demographics and baseline characteristics of patients in the placebo and treatment groups were overall similar (FIG. 5). Nearly all patients (28/30, 95% of Compound 1 and 90% of placebo) completed Part 1 (FIG. 6). Compound 1 (10 mg) was well-tolerated with a similar incidence of AEs as placebo (FIG. 7). All AEs were mild or moderate with no serious AEs or trends in AEs or laboratory abnormalities reported. There were no significant changes in cT1 from baseline to end of treatment (FIG. 9). Compound 1 (10 mg) resulted in >98% inhibition of plasma VAP-1 activity in most subjects by week 2 and sustained suppression through week 12 (FIG. 10). There were no statistically significant differences between placebo and 10 mg Compound 1 in change from BL cT1, liver fat fraction, liver enzymes, or cytokeratin-18. At Week 12, significant differences from placebo in a marker of cell adhesion, VCAM-1, and a marker of hepatic fibrogenesis, tissue inhibitor of metalloproteinase-1 (TIMP-1) were observed (FIGS. 8A, 8B, 11 and 12). Markers of cell adhesion, including VCAM-1 and ICAM-1: VCAM-1, decreased by a mean (±SE) of 24.327 (20.5850) μg/L, in the Compound 1 group and increased by a mean (±SE) of 63.450 (31.6861), in the placebo group relative to BL (p=0.0245) at Week 12. ICAM-1 decreased by a mean (±SE) of 34.85 (56.03) μg/L, in the Compound 1 group and increased by a mean (±SE) of 7.46 (46.97) in the placebo group relative to BL (p=0.0313) at Week 8. At Week 12, tissue TIMP-1, decreased by a mean (±SE) of 29.58 (9.26) ng/ml in the Compound 1 group and increased by a mean (±SE) of 13.56 (14.22) ng/ml in the placebo group relative to BL (p<0.05).

### Conclusions

Compound 1 was well-tolerated with a safety profile similar to placebo in patients with baseline multiparametric MRI and LS values indicative of NASH with at least stage 2 fibrosis. Compound 1 (10 mg) led to near complete inhibition of plasma VAP-1 activity, decreased levels of the hepatic fibrogenesis marker TIMP-1, and statistically significant decrease in the cell adhesion biomarkers, ICAM-1 (at Week 8) and VCAM-1 (at Week 12), compared to placebo. No statistically significant differences were observed between Compound 1 and placebo on other markers of liver inflammation and injury following 12 weeks of treatment. Overall, these data support further assessment of safety and activity of 20 mg Compound 1 in the ongoing Part 2 of the study in NASH patients

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

What is claimed is:

1. A method of treating non-alcoholic steatohepatitis (NASH) in a patient in need thereof, comprising orally administering to the patient a compound of formula: 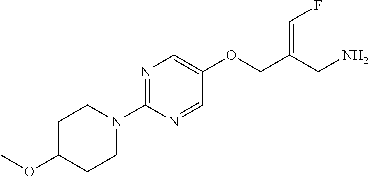 [SMILES: COC1CCN(c2ncc(OC/C(=C/F)CN)cn2)CC1]
or a pharmaceutically acceptable salt thereof once daily at a dose of from about 0.5 mg to about 25 mg.

2. The method of claim 1, wherein the compound, or a pharmaceutically acceptable salt thereof, is administered once daily at a dose of from about 1 mg to about 15 mg.

3. The method of claim 1, wherein the compound, or a pharmaceutically acceptable salt thereof, is administered once daily at a dose of from about 2 mg to about 10 mg.

4. The method of claim 1, wherein the compound, or a pharmaceutically acceptable salt thereof, is administered once daily at a dose of from about 4 mg to about 10 mg.

5. The method of claim 1, wherein the compound, or a pharmaceutically acceptable salt thereof, is administered once daily at a dose of about 1 mg.

6. The method of claim 1, wherein the compound, or a pharmaceutically acceptable salt thereof, is administered once daily at a dose of about 4 mg.

7. The method of claim 1, wherein the compound, or a pharmaceutically acceptable salt thereof, is administered once daily at a dose of about 10 mg.

8. A method of treating NASH in a patient in need thereof, comprising administering to the patient a compound of formula: 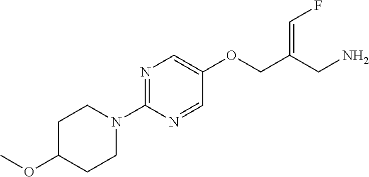 [SMILES: COC1CCN(c2ncc(OC/C(=C/F)CN)cn2)CC1]
or a pharmaceutically acceptable salt thereof at a dose that that results in methylamine concentrations of at least about 25 ng/mL in the plasma when the compound reaches its steady state concentration in the plasma.

9. The method of claim 8, wherein the compound, or a pharmaceutically acceptable salt thereof, is administered at a dose that that results in methylamine concentrations of at least about 50 ng/ml in the plasma when the compound reaches its steady state concentration in the plasma.

10. The method of claim 8, wherein the compound, or a pharmaceutically acceptable salt thereof, is administered at a dose that that results in methylamine concentrations of at least about 100 ng/ml in the plasma when the compound reaches its steady state concentration in the plasma.

11. The method of claim 8, wherein the compound, or a pharmaceutically acceptable salt thereof, is administered at a dose that that results in methylamine concentrations of at least about 300 ng/mL in the plasma when the compound reaches its steady state concentration in the plasma.

12. The method of claim 8, wherein compound, or a pharmaceutically acceptable salt thereof, is administered at a dose that that results in methylamine concentrations of from about 25 ng/mL to about 400 ng/mL in the plasma when the compound reaches its steady state concentration in the plasma.

13. The method of claim 8, wherein compound, or a pharmaceutically acceptable salt thereof, is administered at a dose that that results in methylamine concentrations of from about 50 ng/ml to about 350 ng/mL in the plasma when the compound reaches its steady state concentration in the plasma.

14. The method of claim 8, wherein compound, or a pharmaceutically acceptable salt thereof, is administered at a dose that that results in methylamine concentrations of from about 100 ng/mL to about 300 ng/mL in the plasma when the compound reaches its steady state concentration in the plasma.

15. The method of any one of claims 8 to 14, wherein the compound, or a pharmaceutically acceptable salt thereof, is administered orally.

16. The method of any one of claims 8 to 14, wherein the compound, or a pharmaceutically acceptable salt thereof is administered parenterally.

17. A method of treating NASH in a patient in need thereof, comprising administering to the patient a compound of formula: 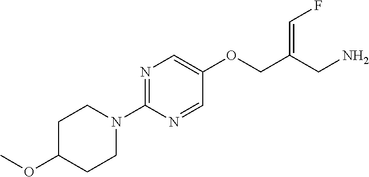 [SMILES: COC1CCN(c2ncc(OC/C(=C/F)CN)cn2)CC1]
or a pharmaceutically acceptable salt thereof at a dose to obtain a steady state area under the curve from zero to infinity (AUC_{0-∞}) of from about 100 ng*h/mL to about 1,000 ng*h/mL.

18. The method of claim 17, wherein the compound, or a pharmaceutically acceptable salt thereof, is administered at a dose to obtain a steady state AUC_{0-∞} of from about 100 ng*h/mL to about 700 ng*h/mL.

19. The method of claim 17, wherein the compound, or a pharmaceutically acceptable salt thereof, is administered at a dose to obtain a steady state AUC_{0-∞} of from about 500 ng*h/mL to about 1,000 ng*h/mL.

20. The method of claim 17, wherein the compound, or a pharmaceutically acceptable salt thereof, is administered at a dose to obtain a steady state AUC_{0-∞} of from about 500 ng*h/mL to about 700 ng*h/mL.

21. The method of any one of claims 17 to 20, wherein the compound, or a pharmaceutically acceptable salt thereof, is administered orally.

22. The method of any one of claims 17 to 20, wherein the compound, or a pharmaceutically acceptable salt thereof, is administered parenterally.

23. A method of treating NASH in a patient in need thereof, comprising administering to the patient a compound of formula: 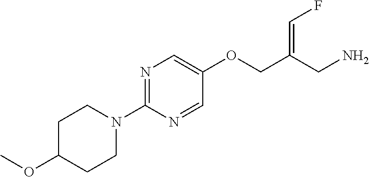 [SMILES: COC1CCN(c2ncc(OC/C(=C/F)CN)cn2)CC1]
or a pharmaceutically acceptable salt thereof, wherein the compound, or pharmaceutically acceptable salt thereof, is administered once every other day.

24. A method of treating NASH in a patient in need thereof, comprising administering to the patient a compound of formula: 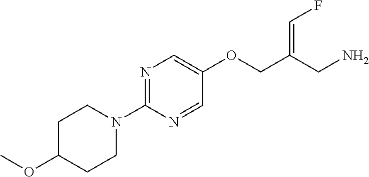 [SMILES: COC1CCN(c2ncc(OC/C(=C/F)CN)cn2)CC1]
or a pharmaceutically acceptable salt thereof, wherein the compound, or pharmaceutically acceptable salt thereof, is administered twice weekly.

25. A method of treating NASH in a patient in need thereof, comprising administering to the patient a compound of formula: 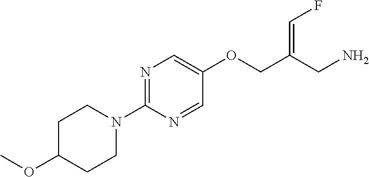 [SMILES: COC1CCN(c2ncc(OC/C(=C/F)CN)cn2)CC1]
or a pharmaceutically acceptable salt thereof, wherein the compound, or pharmaceutically acceptable salt thereof, is administered once weekly.

26. The method of any one of claims 23 to 25, wherein the compound, or pharmaceutically acceptable salt thereof, is administered at a dose of from about 0.5 mg to about 25 mg.

27. The method of claim 26, wherein the compound, or a pharmaceutically acceptable salt thereof, is administered at a dose of from about 1 mg to about 15 mg.

28. The method of claim 26, wherein the compound, or a pharmaceutically acceptable salt thereof, is administered at a dose of from about 2 mg to about 10 mg.

29. The method of claim 26, wherein the compound, or a pharmaceutically acceptable salt thereof, is administered at a dose of from about 4 mg to about 10 mg.

30. The method of claim 26, wherein the compound, or a pharmaceutically acceptable salt thereof, is administered at a dose of about 1 mg.

31. The method of claim 26, wherein the compound, or a pharmaceutically acceptable salt thereof, is administered at a dose of about 4 mg.

32. The method of claim 26, wherein the compound, or a pharmaceutically acceptable salt thereof, is administered once daily at a dose of about 10 mg.

33. The method of any one of claims 23 to 32, wherein the compound, or a pharmaceutically acceptable salt thereof, is administered orally.

34. The method of any one of claims 23 to 32, wherein the compound, or a pharmaceutically acceptable salt thereof is administered parenterally.

35. The method of any one of claims 1 to 34, wherein the compound is administered in combination with a Farnesoid X Receptor (FXR) agonist.

36. The method of claim 35, wherein the FXR agonist is 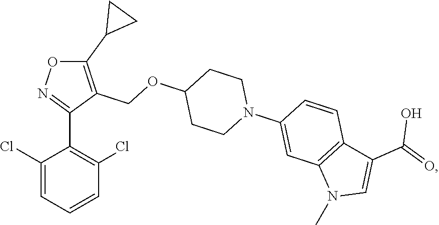 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof.

37. The method of any one of claims 1 to 34, wherein the compound is administered in combination with a peroxisome proliferator-activated receptor (PPAR) agonist.

38. The method of claim 37, wherein the PPAR agonist is pioglitazone, rosiglitazone, elafibranor, saroglitazar, lanifibranor, or seladelpar.

39. The method of any one of claims 1 to 34, wherein the compound is administered in combination with a pan-caspase inhibitor.

40. The method of claim 39, wherein the pan-caspase inhibitor is emricasan.

41. The method of any one of claims 1 to 34, wherein the compound is administered in combination with a galectin-3 inhibitor.

42. The method of claim 41, wherein the galectin-3 inhibitor is belapectin.

43. The method of any one of claims 1 to 34, wherein the compound is administered in combination with a stearoyl Co-A desaturase 1 inhibitor.

44. The method of claim 43, wherein the stearoyl Co-A desaturase 1 inhibitor is armachol.

45. The method of any one of claims 1 to 34, wherein the compound is administered in combination with a chemokine receptor type 2 and 5 (CCR2/CCR5 chemokine) antagonist.

46. The method of claim 45, wherein the CCR2/CCR5 chemokine agonist is cenicriviroc.

47. The method of any one of claims 1 to 34, wherein the compound is administered in combination with an antioxidant.

48. The method of claim 47, wherein the antioxidant is Vitamin E.
